(19) United States
(12) Patent Application Publication
(10) Pub. No.: US 2008/0183225 A1
(43) Pub. Date: Jul. 31, 2008

(54) ORIENTATION-INDEPENDENT IMPLANTABLE ELECTRODE ARRAYS

(76) Inventors: Thomas H. Adamski, Andover, MN (US); Daniel R. Greeninger, Coon Rapids, MN (US); John C. Mertz, Maple Grove, MN (US); Christopher C. Stancer, Prescott, WI (US); James Strom, Arden Hills, MN (US)

Correspondence Address: MEDTRONIC, INC., 710 MEDTRONIC PARKWAY NE, MINNEAPOLIS, MN, 55432-9924, US

(21) Appl. No.: 11/669,378

(22) Filed: Jan. 31, 2007

(51) Int. Cl. A61N 1/05

(52) U.S. Cl. 607/2

(57) ABSTRACT

Apparatus and method according to the disclosure relate to a mechanically and electrically coupling a plurality of electrodes to major opposing surface portions of an implantable medical device (IMD). The surface portions can comprise major opposing surfaces of a connector module of the IMD and/or substantially planar metallic surfaces of the IMD. The electrodes provide a subcutaneous cardiac activity sensing device via the plurality of electrodes which can be used in conjunction with one or more electrodes disposed in an insulative shroud coupled to the peripheral, minor surfaces of the IMD.

### CROSS REFERENCE TO RELATED APPLICATIONS

The present patent document is related to co-pending non-provisional patent applications; namely, Ser. No. 11/085,843, entitled, “APPARATUS AND METHODS OF MONITORING CARDIAC ACTIVITY UTILIZING IMPLANTABLE SHROUD-BASED ELECTRODES,” filed on 22 Mar. 2005 and Ser. No. 11/380,811 entitled, “SHROUD-BASED ELECTRODES HAVING VENTED GAPS,” filed 28 Apr. 2006, the contents of which are hereby fully incorporated by reference herein.

### FIELD OF THE INVENTION

The present invention relates generally to implantable medical devices (IMDs) and more particularly to a subcutaneous multiple electrode sensing and recording system for acquiring electrocardiographic data and waveform tracings from an implanted medical device without the need for or use of surface (skin) electrodes. More particularly, the present invention relates to implantable devices that are equipped with an array of electrodes that operate essentially independent of the final orientation of the IMD following implantation (e.g., they reliably provide adequate far-field electrical sensing of cardiac events).

### BACKGROUND OF THE INVENTION

The electrocardiogram (ECG) is commonly used in medicine to determine the status of the electrical conduction system of the human heart. As practiced the ECG recording device is commonly attached to the patient via ECG leads connected to pads arrayed on the patient's body so as to achieve a recording that displays the cardiac waveforms in any one of 12 possible vectors.

Since the implantation of the first cardiac pacemaker, implantable medical device technology has advanced with the development of sophisticated, programmable cardiac pacemakers, pacemaker-cardioverter-defibrillator arrhythmia control devices and drug administration devices designed to detect arrhythmias and apply appropriate therapies. The detection and discrimination between various arrhythmic episodes in order to trigger the delivery of an appropriate therapy is of considerable interest. Prescription for implantation and programming of the implanted device are based on the analysis of the PQRST electrocardiogram (ECG) that currently requires externally attached electrodes and the electrogram (EGM) that requires implanted pacing leads. The waveforms are usually separated for such analysis into the P-wave and R-wave in systems that are designed to detect the depolarization of the atrium and ventricle respectively. Such systems employ detection of the occurrence of the P-wave and R-wave, analysis of the rate, regularity, and onset of variations in the rate of recurrence of the P-wave and R-wave, the morphology of the P-wave and R-wave and the direction of propagation of the depolarization represented by the P-wave and R-wave in the heart. The detection, analysis and storage of such EGM data within implanted medical devices are well known in the art. For example, S-T segment changes can be used to detect an ischemic episode. Acquisition and use of ECG tracing(s), on the other hand, has generally been limited to the use of an external ECG recording machine attached to the patient via surface electrodes of one sort or another.

The aforementioned ECG systems that utilize detection and analysis of the PQRST complex are all dependent upon the spatial orientation and number of electrodes available in or around the heart to pick up the depolarization wave front

As the functional sophistication and complexity of implantable medical device systems increased over the years, it has become increasingly more important for such systems to include a system for facilitating communication between one implanted device and another implanted device and/or an external device, for example, a programming console, monitoring system, or the like. For diagnostic purposes, it is desirable that the implanted device be able to communicate information regarding the device's operational status and the patient's condition to the physician or clinician. State of the art implantable devices are available which can even transmit a digitized electrical signal to display electrical cardiac activity (e.g., an ECG, EGM, or the like) for storage and/or analysis by an external device. The surface ECG, in fact, has remained the standard diagnostic tool since the very beginning of pacing and remains so today.

To diagnose and measure cardiac events, the cardiologist has several tools from which to choose. Such tools include twelve-lead electrocardiograms, exercise stress electrocardiograms, Holter monitoring, radioisotope imaging, coronary angiography, myocardial biopsy, and blood serum enzyme tests. Of these, the twelve-lead electrocardiogram (ECG) is generally the first procedure used to determine cardiac status prior to implanting a pacing system; thereafter, the physician will normally use an ECG available through the programmer to check the pacemaker's efficacy after implantation. Such ECG tracings are placed into the patient's records and used for comparison to more recent tracings. It must be noted, however, that whenever an ECG recording is required (whether through a direct connection to an ECG recording device or to a pacemaker programmer), external electrodes and leads must be used.

Unfortunately, surface electrodes have some serious drawbacks. For example, electrocardiogram analysis performed using existing external or body surface ECG systems can be limited by mechanical problems and poor signal quality. Electrodes attached externally to the body are a major source of signal quality problems and analysis errors because of susceptibility to interference such as muscle noise, power line interference, high frequency communication equipment interference, and baseline shift from respiration or motion. Signal degradation also occurs due to contact problems, ECG waveform artifacts, and patient discomfort. Externally attached electrodes are subject to motion artifacts from positional changes and the relative displacement between the skin and the electrodes. Furthermore, external electrodes require special skin preparation to ensure adequate electrical contact. Such preparation, along with positioning the electrode and attachment of the ECG lead to the electrode needlessly prolongs the pacemaker follow-up session. One possible approach is to equip the implanted pacemaker with the ability to detect cardiac signals and transform them into a tracing that is the same as or comparable to tracings obtainable via ECG leads attached to surface electrodes.

Previous art describes how to monitor electrical activity of the human heart for diagnostic and related medical purposes. U.S. Pat. No. 4,023,565 issued to Ohlsson describes circuitry for recording ECG signals from multiple lead inputs. Similarly, U.S. Pat. No. 4,263,919 issued to Levin, U.S. Pat. No. 4,170,227 issued to Feldman, et al, and U.S. Pat. No. 4,593,702 issued to Kepski, et al, describe multiple electrode systems, which combine surface EKG signals for artifact rejection.

The primary use for multiple electrode systems in the prior art is vector cardiography from ECG signals taken from multiple chest and limb electrodes. This is a technique whereby the direction of depolarization of the heart is monitored, as well as the amplitude. U.S. Pat. No. 4,121,576 issued to Greensite discusses such a system.

Numerous body surface ECG monitoring electrode systems have been employed in the past in detecting the ECG and conducting vector cardiographic studies. For example, U.S. Pat. No. 4,082,086 to Page, et al., discloses a four electrode orthogonal array that may be applied to the patient's skin both for convenience and to ensure the precise orientation of one electrode to the other. U.S. Pat. No. 3,983,867 to Case describes a vector cardiography system employing ECG electrodes disposed on the patient in normal locations and a hex axial reference system orthogonal display for displaying ECG signals of voltage versus time generated across sampled bipolar electrode pairs.

With regard to various aspects of time-release of surface coatings and the like for chronically implanted medical devices, the following issued patents are incorporated herein by reference. U.S. Pat. Nos. 6,997,949 issued 14 Feb. 2006 and entitled, “Medical device for delivering a therapeutic agent and method of preparation,” and 4,506,680 entitled, “Drug dispensing body implantable lead.” In the former patent, the following is described (from the Abstract section of the '949 patent) as follows: A device useful for localized delivery of a therapeutic agent is provided. The device includes a structure including a porous polymeric material and an elutable therapeutic agent in the form of a solid, gel, or neat liquid, which is dispersed in at least a portion of the porous polymeric material. Methods for making a medical device having blood-contacting surface electrodes is also provided.

Moreover, in regard to subcutaneously implanted EGM electrodes, the aforementioned Lindemans U.S. Pat. No. 4,310,000 discloses one or more reference sensing electrode positioned on the surface of the pacemaker case as described above. U.S. Pat. No. 4,313,443 issued to Lund describes a subcutaneously implanted electrode or electrodes for use in monitoring the ECG. Finally, U.S. Pat. No. 5,331,966 to Bennett, incorporated herein by reference, discloses a method and apparatus for providing an enhanced capability of detecting and gathering electrical cardiac signals via an array of relatively closely spaced subcutaneous electrodes (located on the body of an implanted device).

### SUMMARY

The present invention relates to implantable devices that are equipped with an array of electrodes that operate essentially independent of the final orientation of the IMD following implantation (e.g., they reliably provide adequate far-field electrical sensing of cardiac events). The present invention provides a leadless subcutaneous (or submuscular) electrode array that, once implanted, provides a variety of sensing vectors, including vectors incorporating signals from at least one electrode disposed on a major planar surface of an IMD.

For example, in one embodiment a compliant electrically insulative member couples to a major surface of an IMD, such as a lateral side of a header module and/or major planar surfaces of the IMD. An optional mechanical barb, or boss member, can project from the IMD housing to mechanically engage the insulative member. The insulative member mechanically supports at least one electrode, which can comprise a substantially planar electrode. In one embodiment three such electrodes are mechanically coupled to a first side of an IMD. In another embodiment electrodes couple to opposing major sides of the IMD. Herein such electrodes are referred to as the “major surface electrodes” to distinguish same from other electrodes disposed within a shroud member that couples to a part of the periphery of IMD. In yet a third embodiment, discrete electrodes couple to the shroud member and at least one major surface of the IMD.

The major surface electrodes can include elongated insulated conductor routed to the header portion of the IMD (and to the hermetic feedthrough pins to pass signals to internal circuitry) or can route signals directly through a dedicated monopolar feedthrough adjacent to or under each major surface electrode. The major surface electrodes are electrically insulated from the typically metallic IMD housing and can be hermetically coupled through the housing with any of a variety of types of known construction. For example, the feedthrough can comprise a glass-to-metal seal with a conductive pin sealed therein, a brazed seal, a ceramic or organic, a polymeric-compression feedthrough and the like. For electrical insulation from the housing one of more mechanical stand-offs, barbs, or boss members can be disposed to engage an insulative biocompatible adhesive such as Tecothane which in turn couples to a major surface electrode. In lieu of this technique, a patch of adhesive tape can be used and/or a dielectric material coated or layered on the face of the electrode abutting the metallic IMD housing. The major surface electrodes can be configured in a wide variety of shapes and sizes, including so-called integrated nailhead pin (or flattened post) or simple potted-pin feedthroughs that function as both a feedthrough and an electrode.

The electrode can comprise a mesh screen-type, a plate, a coil—or spiral—electrode (particularly if the both ends of the coil is firmly connected to structure so it does not tend to uncoil), simple and the like. The major surface feedthroughs can be coated with platinum black, titanium nitride and the like.

The major surface electrodes can be substantially planar, convex or concave in cross section and having a surface area that varies as a function of the surface areas of the other electrodes coupled to the IMD. To reduce electrical noise, such as from myopotentials from nearby pectoral muscles, the major surface electrodes can be disposed distributed across a common major surface of an IMD and facing the skin (not the pectoral muscle) upon implant. This configuration provides adequate far-field sensing of cardiac activity while reducing such undesirable noise.

The major surface electrodes (and, if applicable, shroud-type) electrically couple to circuitry of an IMD and are adapted to detect cardiac activity of a subject. Temporal recordings of the detected cardiac activity are referred to herein as an extra-cardiac electrogram (EC-EGM). The recordings can be stored upon computer readable media within an IMD at various resolution (e.g., continuous beat-by-beat, periodic, triggered, mean value, average value, etc.). Real time or stored EC-EGM signals can be provided to remote equipment via telemetry. For example, when telemetry, or programming, head of an IMD programming apparatus is positioned within range of an IMD the programmer receives some or all of the EC-EGM signals.

The present invention provides improved apparatus and methods for reliably collecting EC-EGM signals for use or collection in conjunction with diverse IMDs (e.g., implantable pacemakers having endocardial leads, implantable cardioverter-defibrillators or ICDs, drug delivery pumps, subcutaneous ICDs, submuscular ICDs, brain stimulation devices, nerve stimulation devices, physiologic monitors, muscle stimulation devices and the like).

The invention can be implemented employing suitable sensing amplifiers, switching circuits, signal processors, and memory to process the EC-EGM signals collected between any selected pair or pairs of the major surface electrodes and/or shroud-based electrodes deployed in an array across and/or around a housing of an IMD to provide a leadless, orientation-insensitive means for receiving the EC-EGM signals from the heart.

Each of the electrically insulative members that support the major surface electrode(s) and, as applicable, the shroud member can be fabricated of a non-conductive, bio-compatible material such as any appropriate resin-based material, urethane polymer, silicone, or relatively soft urethane that retains its mechanical integrity during manufacturing and prolonged exposure to body fluids. These materials are mechanically and/or chemically adhered to the portions of an IMD in a number of configurations (e.g., two, three, four electrodes) for individual electrodes. However, a three-electrode embodiment appears to provide an improved signal-to-noise ratio than the other electrode configuration; especially if they are arranged at approximately 120 degrees from each other.

Embodiments having electrodes connected to three sense-amplifiers that are hardwired to three electrodes record simultaneous EC-EGM signals. Alternative embodiments employ electrodes on the face of the lead connector, or header module, and/or major planar face(s) of the pacemaker that may be selectively or sequentially coupled in one or more pairs to the terminals of one or more sense amplifiers to pick up, amplify and process the EC-EGM signals across each electrode pair. In one aspect, the EC-EGM signals from a first electrode pair are stored and compared to signals from other electrode pair(s) in order to determine the optimal sensing vector. Following such an optimization procedure, the system can be programmed to chronically employ the selected subcutaneous EC-EGM signal vector or to automatically compare the sensing vectors to maximize EC-EGM sensing.

For mass production of assemblies according to the invention a unique electrode piecepart can be fabricated for each unique conductor pathway and configuration (including any of the variety of diverse mechanical interlocking features between electrode and insulative material and/or shroud). Besides manufacturing processes such as metal stamping, the metallic electrode member(s) can be fabricating using electron discharge machining (EDM), laser cutting, or the like. It is desirable that the electrode assemblies are pre-configured (at least in a two-dimensional manner) so that little or no mechanical deformation or bending is required to assemble them. In addition, if parts are pre-configured, the parts can bent in a predictable manner and retain relatively little, if any, energy due to the spring-constant of the metal used to form the parts. In the event that electrical insulation or a dielectric layer becomes necessary or desirable, all or a portion of the electrode assembly can be coated with an insulative material such as paralyne or similar while the portions of the assembly likely to contact body fluid can be coating with diverse coatings pursuant to various embodiments of the invention.

Electrode assemblies according to the invention can be used for chronic or acute extra-cardiac electrogram (EC-EGM) signal sensing collection and attendant heart rate monitoring, capture detection, arrhythmia detection, and the like as well as detection of myriad other cardiac insults (e.g., ischemia monitoring using S-T segment changes, pulmonary edema monitoring based upon impedance changes).

In addition, the surface of the electrodes can be treated with one or more electrode coatings to enhance signal-conducting, de- and re-polarization sensing properties, and to reduce polarization voltages (e.g., platinum black, titanium nitride, titanium oxide, iridium oxide, carbon, etc.). That is, the surface area of the electrode surfaces may be increased by techniques known in the art and/or can be coated with such materials as just described and equivalents thereof. All of these materials are known to increase the true electrical surface area to improve the efficiency of electrical performance by reducing wasteful electrode polarization, among other advantages.

These and other advantageous aspects of the invention will be appreciated by those of skill in the art after studying the invention herein described, depicted and claimed. In addition, persons of skill in the art will appreciate insubstantial modifications of the invention that are intended to be expressly covered by the appended claims.

### BRIEF DESCRIPTION OF THE DRAWINGS

FIG. 1 is an elevational side view depicting an exemplary shroud assembly coupled to an IMD which illustrates electrical conductors disposed in the header, or connector, portion of the IMD which is configured to receive a proximal end portion of medical electrical leads (not shown).

FIG. 2 is a perspective view of the IMD depicted in FIG. 1 further illustrating the shroud assembly.

FIG. 3 is a perspective view of an opposing major side of the IMD depicted in FIGS. 1 and 2.

FIG. 4 is a plan view of the IMD previously depicted that illustrates the relationship between two of the electrodes coupled to the shroud assembly as well as depicting the header, or connector, of the IMD.

FIG. 5 is a photocopy copy of a first side of a transparent shroud assembly coupled to a header according to the invention that clearly illustrates that the conductors and components of the assembly are readily visible.

FIG. 6 is a photocopy copy of a second side of the transparent shroud assembly coupled to a header according to the invention that clearly illustrates that the conductors and components of the assembly are readily visible from both sides.

FIG. 7 is a plan view of an IMD having an array of three major surface electrodes spaced apart and coupled to a common surface of the IMD.

FIG. 8 is a perspective view of an IMD having both a compliant shroud with embedded electrodes and an array of three major surface electrodes spaced apart and coupled to a common surface of the IMD.

### DETAILED DESCRIPTION OF THE DRAWINGS

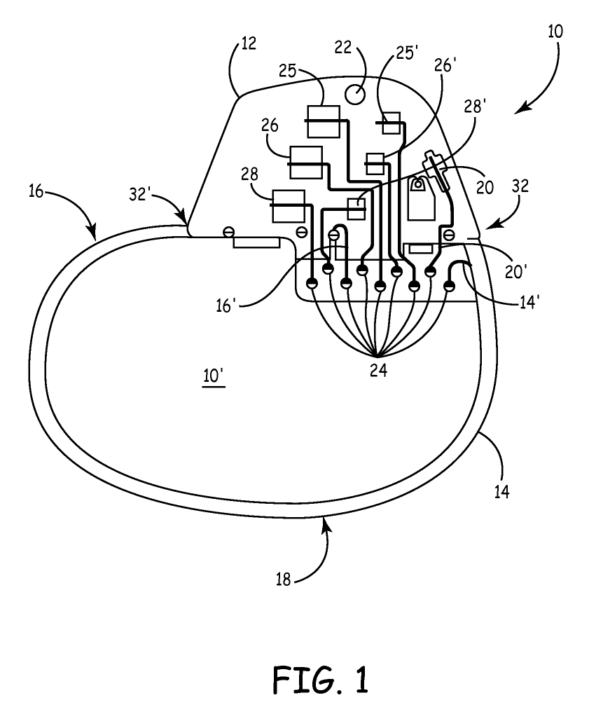

FIG. 1 is an elevational side view depicting an exemplary shroud assembly 14 coupled to an IMD 10 which illustrates electrical conductors 24,25,26,28 disposed in the header, or connector, portion 12 of the IMD 10 which are configured to couple to end portions of medical electrical leads as well as couple to operative circuitry within the IMD housing (not shown). The shroud assembly 14 surrounds IMD 10 and mechanically couples to the header portion 12 and includes at least three discrete electrodes 16,18,20 adapted for sensing far-field, or extra-cardiac electrogram (EC-EGM) signals. FIG. 1 also depicts an aperture 22 formed within the header 12 which can be used to receive thread used to suture the header 12 (and thus the IMD 10) to a fixed surgical location (also known as a pocket) of a patient's body.

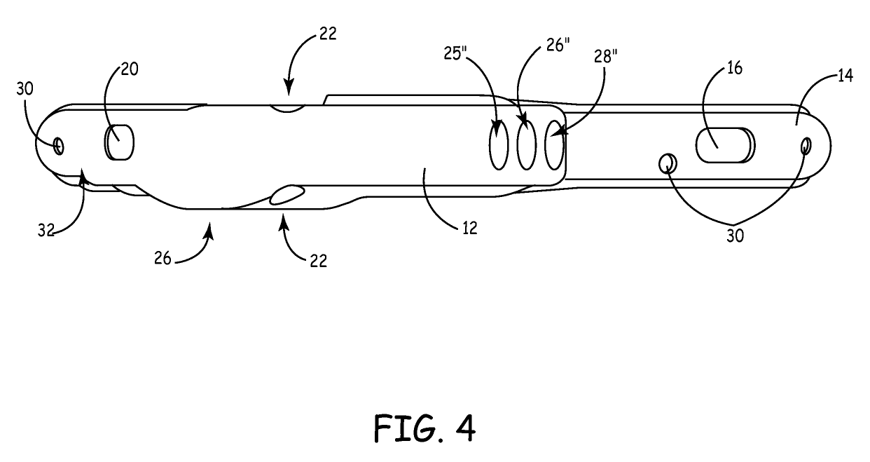

As partially depicted in FIG. 1, an elongated conductor 14′ couples to electrode 14, elongated conductor 16′ couples to electrode 16, and conductor segment 20′ couples to electrode 20. Furthermore, three of the conductors (denoted collectively with reference numeral 24) couple to three cuff-type conductors 25,26,28 adapted to receive proximal portions of medical electrical leads while another three of the conductors couple to conductive pads 25′,26′,28′ which are aligned with, but spaced from the conductors 25,26,28 along a trio of bores (denoted as 25″,26″,28″ in FIG. 4 herein) formed in header 12.

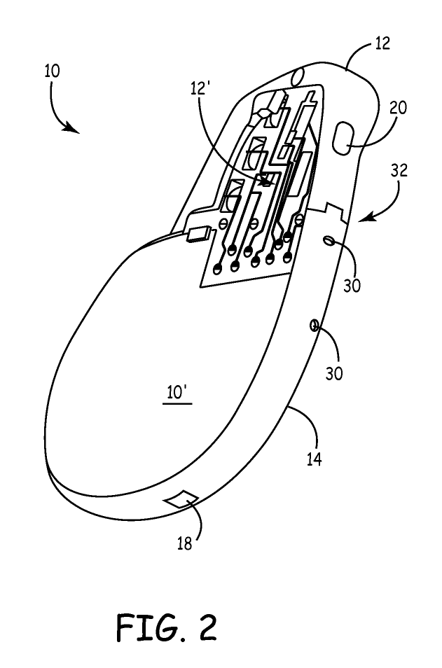

FIG. 2 is a perspective view of the IMD 10 depicted in FIG. 1 further illustrating the shroud assembly 14 and two of the three electrodes 18,20. In addition, two of a plurality of adhesive ports 30 and a mechanical joint 32 between the elongated portion of the shroud assembly 14 and the header 12 are also depicted in FIG. 2. The ports 30 can be used to evacuate excess medical adhesive disposed between the shroud assembly 14 and the IMD 10 and/or used to inject medical adhesive into one or more ports 30 to fill the void(s) therebetween. In one form of the invention, a major lateral portion 12′ of header 12 remains open to ambient conditions during assembly of the IMD 10. Subsequent to making electrical connections between the plurality of conductors of the shroud assembly 14 and the header 12, the open lateral portion 12′ is sealed (e.g., automatically or manually filled with a biocompatible substance such as a substantially clear medical adhesive, such as Tecothane® made by Noveon, Inc. a wholly owned subsidiary of The Lubrizol Corporation). Thus most if not all of the plurality of conductors of the shroud assembly 14 and the IMD 10 are visible and can be manually and/or automatically inspected to ensure long term operability and highest quality of the completed IMD 10.

Some properties of various Tecothane® appear below (as published in the Technical Data Sheet (TDS) for certain clear grades of the material:

| | ASTM Test | TT-1074A | TT-1086A | TT-1096A | TT-1066D | TT-1065D | TT-1069D | TT-1072D | TT-1076D-M |
|---|---|---|---|---|---|---|---|---|---|
| Durometor | D2240 | 75A | 85A | 94A | 94D | 64D | 69D | 74D | 75D |
| (Store Hradness) | | | | | | | | | |
| Speciflc Gravity | D792 | 1.10 | 1.12 | 1.15 | 1.16 | 1.18 | 1.18 | 1.18 | 1.19 |
| Flexural Mudulus | D700 | 1.300 | 3.000 | 8.000 | 18.000 | 26.000 | 44.000 | 73.000 | 180.000 |
| (psi) | | | | | | | | | |
| Ultimate Tensile | D412 | 6.000 | 7.000 | 9.000 | 9.000 | 10.000 | 8.800 | 9.000 | 8.300 |
| (psi) | | | | | | | | | |
| Ultimate Elongation | D412 | 550 | 450 | 400 | 350 | 300 | 310 | 279 | 150 |
| (%) | | | | | | | | | |
| Tensile (psi) | D412 | | | | | | | | |
| at 100% Elongation | | 500 | 800 | 1.300 | 2.500 | 2.800 | 3.200 | 3.700 | 3.600 |
| at 200% Elongation | | 700 | 1.000 | 2.100 | 3.800 | 4.600 | 4.200 | 3.900 | NA |
| at 300% Elongation | | 1.100 | 1.600 | 4.300 | 6.500 | 7.600 | NA | NA | NA |
| Melt Index | D1228 | 3.5 | 4.0 | 3.8 | 4.0 | 2.0 | 3.0 | 2.0 | 5.0 |
| (gm/10 min at | | (205° C.) | (205° C.) | (210° C.) | (210° C.) | (210° C.) | (210° C.) | (210° C.) | (210° C.) |
| 2190 gm/load) | | | | | | | | | |
| Mold shrinkage (in/in) | D855 | 008–012 | 008–012 | 006–010 | 004–008 | 004–008 | 004–008 | 004–006 | 004–006 |

Referring again to FIG. 2, the terminal ends of conductors 24 are depicted to include the optional shaped-end portion which provides a target for reliable automatic and/or manual coupling (e.g., laser welding, soldering, and the like) of the terminal end portions to respective conductive pins of a multi-polar feedthrough assembly (not shown). As is known in the art, such conductive pins hermetically couple to operative circuitry disposed within the IMD 10.

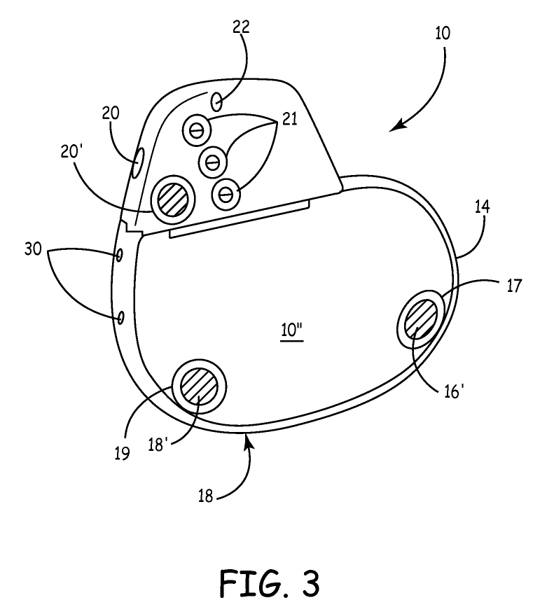

FIG. 3 is a perspective view of an opposing major side 10″ of the IMD 10 depicted in FIGS. 1 and 2 and three self-healing grommets 21 substantially hermetically coupled to openings of a like number of threaded bores (shown in FIG. 6 and denoted by reference numeral 26′). As is known, the threaded bores are configured to receive a threaded shank and the grommets 21 are fabricated to temporarily admit a mechanical tool (not shown). The tool is used to connect and allow a physician or clinician to manually tighten the conductors 25,26,28, for example, with compression and/or radially around conductive rings disposed on proximal portions of medical electrical leads (not shown). The IMD 10 also includes a pair of major surface electrodes 16′,18′ spaced apart on surface 10″. The major surface electrodes 16′,18′ can be disposed in a recessed region and/or supported by a layer of insulative material 17,19, respectively. The major surface electrodes 16′,18′ electrically couple to amplifier and filtering circuitry within the IMD 10 via a feedthrough or the like hermetically coupled within the surface 10″ and surrounded by material 17,19. Another major surface electrode 20′ is disposed in a major side surface of the header 12. Since header 12 oftentimes is fabricated of polymer, the electrode 20′ does not necessarily require an analog to material 17,19 of electrodes 16′,18′. Thus, the shroud-based electrodes 18,20 can be sampled in various combinations with the major surface electrodes 16′,18′,20′ and the sensing vector corresponding to the best EC-EGM used to chronically collect cardiac activity signals. In addition, two of the plurality of ports 30 of the shroud member 14 are also depicted in FIG. 3.

FIG. 4 is a plan view of the IMD 10 previously depicted that illustrates the relationship between two of the electrodes 16,20 coupled to the shroud assembly 14 as well as depicting the header 12, or connector, of the IMD 10. Opposing openings of the aperture 22 formed in the header 12 are also depicted in FIG. 4 as are the three openings 25″,26″,28″ of the bores or ports formed in the header 12 that are configured to admit the proximal end of medical electrical leads (not shown). Three of the adhesive-admitting ports 30 are shown distributed at various locations through the surfaces of the shroud 14.

Three elongated conductors individually couple to a respective electrode 14,16,18. These elongated conductors can be continuous or discrete segments of conductive material. In the event that they comprise discrete segments, they need to be coupled together such as with convention means like laser bonding, welding, soldering and the like. For example, the elongated conductor coupling to electrode 16 can traverse either direction around the periphery of the IMD 10 disposed within or mechanically coupled to an inner portion of the shroud 14. If it traverses past the seam 32 it might need to be isolated from the elongated conductor coupled to electrode 18 (assuming that conductor also traversed seam 32). If the conductor coupling electrode 16 is routed directly toward the header 12 (and the header/shroud is not a unitary structure) then a bond between segments of the elongated conductor could be necessary at the junction of the shroud 14 and the header 12.

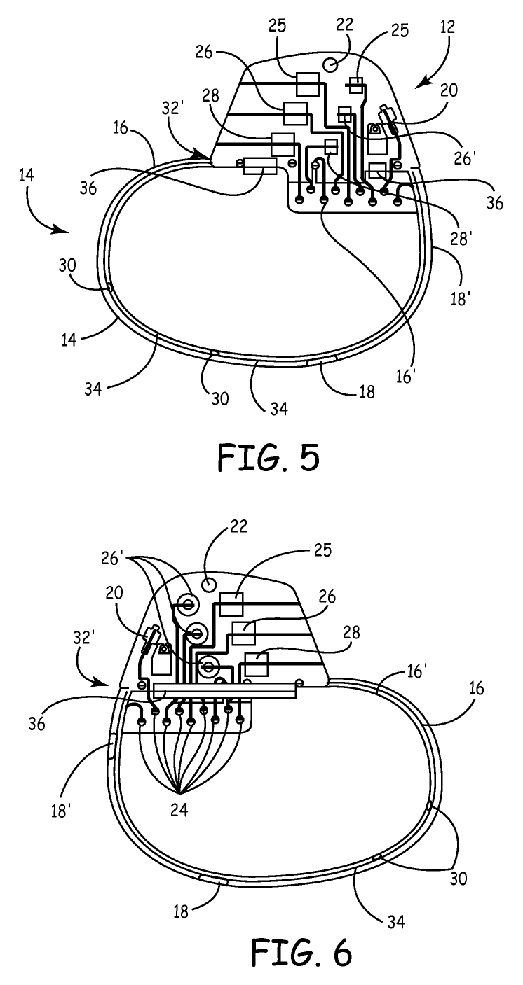

FIG. 5 is a photocopy copy of a first side of a transparent shroud assembly 14 coupled to a header 12 according to the invention that clearly illustrates that the conductors and components of the assembly are readily visible. FIG. 6 is a photocopy copy of a second side of the transparent shroud assembly coupled to a header according to the invention that clearly illustrates that the conductors and components of the assembly are readily visible from both sides.

Since FIG. 5 and FIG. 6 essentially depict common components of the inventive assembly of the invention they shall be described together. The exemplary shroud assembly 14 of FIGS. 5 and 6 is depicted with an IMD 10 for clarity. The electrical conductors 25,26,28 disposed in the header, or connector, portion 12 of the IMD 10 are configured to couple to end portions of medical electrical leads as well as couple to operative circuitry within the IMD housing (not shown). The shroud assembly 14 mechanically couples to the header portion 12 at each end of the shroud assembly 14 both mechanically and electrically via medical adhesive (disposed at overlapping joint 32′) and an elongate conductor 16′ (passing through joint 32′). The three discrete electrodes 16,18,20 and their corresponding elongated conductors 16′,18′, 20′ are coupled together. While not depicted in FIGS. 5 and 6 the conductors 16′,18′,20′ have at least a partially serpentine configuration and conductors 16′,18′ are furthermore mechanically coupled to the shroud with a series of elongated stand-off bosses 34. In addition, and as previously mentioned, during attachment to an IMD adhesive is disposed intermediate the shroud 14 and the IMD with excess being evacuated from ports 30 (and/or if needed injected into one of more ports 30) to eliminate any air bubbles. Of course, one feature of the invention relates to the ability to fully inspect the finished article visually (including the quality of the electrical connections and the quality of the bond between the shroud 14 and an IMD. Also, the electrodes 16,18 can be at least one of mechanically embedded partially into the material of the shroud 14 and configured to receive medical adhesive to retain the electrodes in position (e.g., using perforated wing-like peripheral portions of the electrodes disposed at the ends, sides, and/or other parts of the periphery of an electrode). Aperture 22 also can be seen in FIGS. 5 and 6 formed in a peripheral portion of the header 12. Also depicted is how the elongated conductor 14′ couples to electrode 14, elongated conductor 16′ couples to electrode 16, and conductor segment 20′ couples to electrode 20. Furthermore, three of the conductors (denoted collectively with reference numeral 24) couple to three cuff-type conductors 25,26,28 adapted to receive proximal portions of medical electrical leads while another three of the conductors couple to conductive pads 25′,26′,28′ which are aligned with, but spaced from the conductors 25,26,28 along a trio of bores (denoted as 25″,26″,28″ in FIG. 4 herein) formed in header 12. The joint 32 between header 12 and shroud 14 can comprise a variety of mechanisms, including an interlocking, partially spring-biased socket-type connection which, in combination with medical adhesive, provides a reliable mechanical coupling.

Another feature of the invention relates to including radio-opaque markers and/or identifiers within and/or on the shroud 14 so that a physician or clinician can readily determine that an IMD is outfitted with an assembly according to this invention. A marker according to this aspect of the invention can include a metallic insert and/or coating having a unique shape, location and/or configuration (e.g., an “M” or the corporate logo for an IMD manufactured by Medtronic, Inc.).

Depicted in FIGS. 5 and 6 is an elongated structural support member 36 which provides a reliable connection to a metallic housing of an IMD (not shown) via traditional processes (e.g., laser welding). The member 36 has a three substantially orthogonal sides (all denoted as 36 in FIGS. 5 and 6) thus providing three discrete bonding areas between the header 12 and an IMD. Of course, the member 36 could be perforated and/or coated with an insulative material, but in the embodiment depicted one side is cut out or not present so that the plurality of conductors 24 can pass from the header 12 and shroud 14 to the feedthrough array of the IMD.

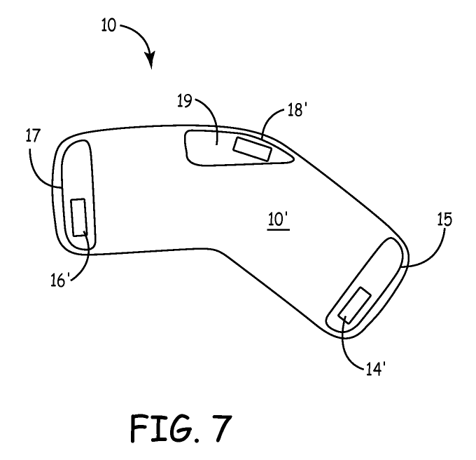

FIG. 7 is a plan view of an IMD 10 having an array of three major surface electrodes 16′,18′,20′ spaced apart and coupled to a common surface 10′ of the IMD 10. Each of said electrodes 14′,16′,18′ are electrically isolated from the IMD with a biocompatible resilient material 15,17,19. Optionally, a barb or boss formed on the IMD abutting the material 15,17,19 can help secure the material, and as a result the electrodes to the IMD. In addition, a monopolar feedthrough can be used to mechanically and electrically couple each electrode to circuitry within the IMD 10. In the alternative, an elongated flexible circuit can be used to route the signals from the electrodes to a common location (e.g., a mass termination at a multipolar feedthrough array) as is known in the art and as described and depicted elsewhere herein.

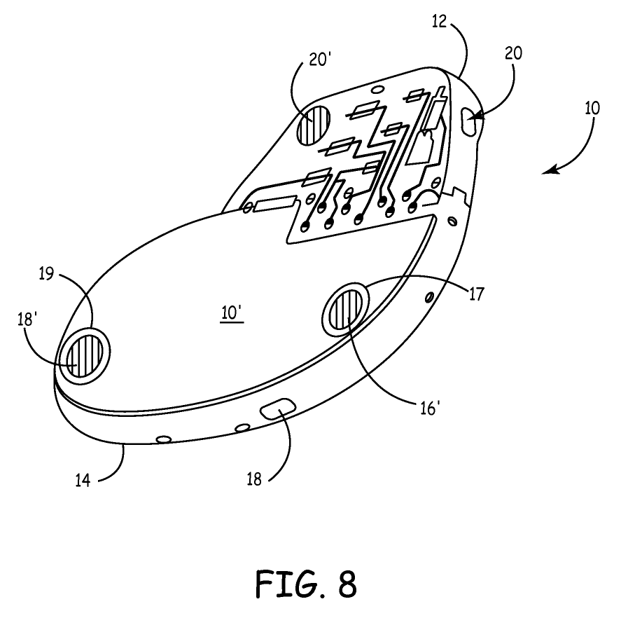

FIG. 8 is a perspective view of an IMD having both a compliant shroud 14 with an embedded electrode 18, a header 12 with another electrode 20, and an array of three major surface electrodes 16′,18′,20′ spaced apart and coupled to a common surface 10′ of the IMD 10. The electrodes 16′,18′ are insulated from the IMD 10 and supported by resilient material 17,19, respectively. Various combinations of sensing pairs of electrode can be tested and signals compared as previously described and the electrodes can be mechanically coupled through the surface 10′ and header 12 as previously described.

The electrodes and/or the (corresponding elongated conductors) can be fabricated out of any appropriate material, including without limitation tantalum, tantalum alloy, titanium, titanium alloy, platinum, platinum alloy, or any of the tantalum, titanium or platinum group of metals whose surface may be treated by sputtering, platinization, ion milling, sintering, etching, or a combination of these processes to create a large specific surface area. Also as noted herein, an electrode can be stamped, drawn, laser cut or machined using electronic discharge apparatus. Some of the foregoing might require de-burring of the periphery of the electrode or alternately any sharp edges due to a burr can be coupled facing toward the corresponding recess in the shroud member thereby minimizing likelihood of any patient discomfort post-implant while further reducing complexity in the fabrication of assemblies according to the invention. The electrodes can be coated or covered with platinum, a platinum-iridium alloy (e.g., 90:10), platinum black, titanium nitride or the like.

Accordingly, a number of embodiments and aspects of the invention have been described and depicted although the inventors consider the foregoing as illustrative and not limiting as to the full reach of the invention. That is, the inventors hereby claim all the expressly disclosed and described aspects of the invention as well as those slight variations and insubstantial changes as will occur to those of skill in the art to which the invention is directed. The following claims define the core of the invention and the inventors consider said claims and all equivalents of said claims and limitations thereof to reside squarely within their invention.

1. A subcutaneous cardiac activity sensing device, comprising:
an implantable medical device (IMD) having opposing major exterior surfaces; and
at least a pair of electrodes mechanically electrically insulated from and coupled to the opposing major exterior surfaces of the IMD;

2. A device according to claim 1, wherein the electrodes are disposed on a common side of the opposing major exterior surfaces of the IMD.

3. A device according to claim 1, wherein the opposing major exterior surfaces of the IMD comprise a metallic material.

4. A device according to claim 3, wherein the metallic material comprises one of a titanium material and a tantalum material.

5. A device according to claim 3, further comprising an insulating material disposed between the electrodes and the opposing major exterior surfaces of the IMD.

6. A device according to claim 5, wherein the insulating material comprises a biocompatible medical adhesive material.

7. A device according to claim 5, wherein the electrodes comprise one of a platinum material, an iridium material, a titanium material.

8. A device according to claim 1, wherein the electrodes further include a coating on at least a major surface thereof.

9. A device according to claim 8, wherein the coating comprises one of a nitride coating, a carbon black coating.

10. A device according to claim 1, further comprising a resin-based connector module coupled to a peripheral portion of the IMD, and wherein the electrodes are disposed solely on said connector module.

11. A device according to claim 2, wherein the electrodes comprise three electrodes spaced apart on the common side.

12. A device according to claim 11, wherein the electrodes are spaced apart at about 120 degrees from each other.

13. A device according to claim 12, wherein each electrode is approximately equally spaced apart from each other electrode.

14. A device according to claim 12, further comprising a monopolar feedthrough assembly hermetically sealed to the common side between at least one of the electrodes and electronic circuit means disposed within the IMD.

15. A device according to claim 13, wherein the feedthrough assembly includes an elongated conductive pin electrically coupling said at least one of the electrodes and the electronic circuit means.

16. A device according to claim 15, further comprising a coating disposed on the electrodes.

17. A device according to claim 1, wherein the coating comprises one of a nitride coating, a carbon black coating, a time-release coating.

18. A device according to claim 1, wherein the IMD comprises one of: an implantable cardiac pacemaker, an implantable cardioverter-defibrillator, an implantable fluid delivery device, an implantable neurostimulator, an implantable gastric simulator.

19. A method of fabricating a cardiac sensing shroud assembly, comprising:
providing an implantable medical device (IMD);
forming at least three apertures in a substantially flat portion of a major surface of the IMD;
coupling at least three conductive electrodes into engagement in each said aperture;
insulating the electrodes from contact with conductive surface portions of the IMD; and
electrically coupling said electrodes to circuitry disposed within the IMD.

20. A method according to claim 19, wherein the IMD comprises one of: an implantable cardiac pacemaker, an implantable cardioverter-defibrillator, an implantable fluid delivery device, an implantable neurostimulator, an implantable gastric simulator.
